(19) United States
(12) Patent Application Publication
(10) Pub. No.: US 2023/0112911 A1
(43) Pub. Date: Apr. 13, 2023

(54) METHOD FOR CALCULATING INTERACTION BETWEEN FEATURE AMOUNTS AND SYSTEM FOR CALCULATING INTERACTION BETWEEN FEATURE AMOUNTS

(71) Applicant: Hitachi High-Tech Corporation, Tokyo (JP)

(72) Inventor: Makiko YOSHIDA, Tokyo (JP)

(21) Appl. No.: 17/948,460

(22) Filed: Sep. 20, 2022

(30) Foreign Application Priority Data
Sep. 28, 2021 (JP) 2021-157769

(51) Int. Cl. G06N 5/04; G06K 9/62; G06N 5/00; G16H 50/30

(52) U.S. Cl. CPC G06N 5/045; G06K 9/6282; G06N 5/003; G16H 50/30

(57) ABSTRACT

System and method for calculating interaction between feature amounts, including a model construction unit for acquiring data including a feature amount vector which is a set of numerical values of feature amounts as an explanatory variable, and information of an event as an objective variable, and constructing a classification and prediction model having a tree structure for classifying and predicting the event based on the feature amount vector, an interaction score calculation unit for calculating an interaction score indicating a degree of association of interaction between the feature amounts with the event is based on a position of the feature amount appearing in a node constituting the classification and prediction model, and a position of the feature amount in the classification and prediction model in which the position of the feature amount appearing in the node has been shuffled, and an output processing unit for outputting the calculated interaction score.

### BACKGROUND OF THE INVENTION

### 1. Field of the Invention

The present invention relates to techniques for a method for calculating an interaction between feature amounts and a system for calculating an interaction between feature amounts.

### 2. Description of Related Art

Research on the human gut microbiota using metagenomic analysis technology has attracted a great deal of international attention. One of the main reasons for this is that it has become clear that there is a close relationship between the human gut microbiota and disease. For example, it has been reported that in addition to colon-related diseases such as pseudomembranous colitis, obesity, diabetes, various autoimmune diseases, colon cancer, liver cancer, renal failure, heart failure, nervous system diseases, mental and brain functions such as autism, which are related to lifestyle and eating habits, are associated with the human gut microbiota. Thus, recent studies have revealed that the structure of the gut microbiota is involved in the systemic function regardless of the organ. By paying attention to the relationship between the gut microbiota and the disease, it is expected that new treatments and preventions different from the conventional ones will be possible for various diseases.

The gut microbiota has a very complicated flora structure in which a large number of bacterial species interact with each other, and interacts with the health condition of the host and the nutrients ingested by the host to affect the physiological function of the host. As a result, the gut microbiota is believed to be involved in the development of various diseases. Therefore, when analyzing the association between gut microbiota and disease, it is important to consider the interaction between many factors including external factors such as health status and nutrient intake, in addition to the factors inside the gut microbiota. Traditional statistical methods are often used in the association analysis in gut microbiota studies. However, since multiple tests are a problem when dealing with a large number of factors in traditional statistical methods, machine learning methods that are excellent in analyzing a large number of factors and interactions thereof have been attracting attention in recent years.

JP-T520510 discloses a pharmacological phenotypic prediction platform for individuals and cohorts, in which “in patients who have or may have a primary or comorbid disease, pharmacological phenotypes can be predicted by a collection of panomix data, physiomics data, environmental data, sociomix data, demographic data, and outcome phenotypic data over a certain period of time. The machine learning engine generates statistical models based on training data from patients for the training, thereby pharmacological phenotypes can be predicted, including drug response and administration, adverse drug events, disease and comorbid disease risk, drug-gene interactions, drug-drug interactions, and multidrug therapy interactions. Then, to benefit from additional predictive power, the model is applied to new patient data to predict pharmacological phenotypes thereof and allow clinical and research decision-making including drug selection and dosages, changing dosing regimens, optimizing multidrug therapy, monitoring, and the like to benefit from additional predictive power, thereby avoiding adverse events and substance abuse, improving drug response, bringing better patient outcomes, lower treatment costs, and public health benefits, and increasing the effectiveness of research in the pharmacology and other biomedical fields” (see abstract).

### SUMMARY OF THE INVENTION

However, in the method presented in JP-T-2020-520510, the machine learning model is used to predict the pharmacological phenotype of the patient based on the data of the new patient. Therefore, it is not possible to extract important factors in the prediction of pharmacological phenotype from the model.

The present invention was made in view of such a background and an object of the present invention is to easily grasp the association between feature amounts and events.

In order to solve the above-mentioned problems, the present invention is characterized in that an arithmetic device executes a model construction step of acquiring data including a feature amount vector which is a set of numerical values of feature amounts and is an explanatory variable, and information of an event which is an objective variable, and constructing a classification and prediction model having a tree structure for classifying and predicting the event based on the feature amount vector, an interaction score calculation step of calculating an interaction score in which a degree of association of an interaction between the feature amounts with the event is scored based on a position of the feature amount appearing in a node constituting the classification and prediction model, and a position of the feature amount in the classification and prediction model in which the position of the feature amount appearing in the node has been shuffled, and an output step of outputting the calculated interaction score to an output unit.

Other solutions will be described as appropriate in the embodiments.

According to the present invention, the association between the feature amount and the event can be easily grasped.

### BRIEF DESCRIPTION OF THE DRAWINGS

FIG. 1 is a diagram showing a configuration example of an arithmetic system according to the embodiment;

FIG. 2 is a flowchart showing a procedure of overall processing performed in a first embodiment;

FIG. 3 is a diagram showing an example of training data in the first embodiment;

FIG. 4 is a flowchart showing a procedure of interaction score calculation processing executed in the first embodiment;

FIG. 5A is a diagram (No. 1) showing a part of a decision tree obtained as a result of applying a random forest to the training data;

FIG. 5B is a diagram (No. 2) showing a part of the decision tree obtained as a result of applying the random forest to the training data;

FIG. 5C is a diagram (No. 3) showing a part of the decision tree obtained as a result of applying the random forest to the training data;

FIG. 6 is a diagram showing an example of a calculation result of an interaction score for a combination of two feature amounts;

FIG. 7 is a diagram showing an example of an output screen in the first embodiment;

FIG. 8 is a diagram showing an example of an output screen in a second embodiment; and

FIG. 9 is a diagram showing an example of training data in a third embodiment.

### DESCRIPTION OF EMBODIMENTS

Next, modes for carrying out the present invention (referred to as “embodiments”) will be described in detail with reference to the drawings as appropriate. However, the present embodiment is merely an example to implement the present invention and does not limit the present invention.

### First Embodiment

The first embodiment shows an example of extracting an interaction associated with pollinosis in an analysis of the association among the gut microbiota, ingested nutrients, and the presence or absence of pollinosis. In the first embodiment and the second embodiment, the presence or absence of pollinosis is used as the objective variable, but the objective variable is not limited to the presence or absence of pollinosis as long as it can be classified.

### System Configuration

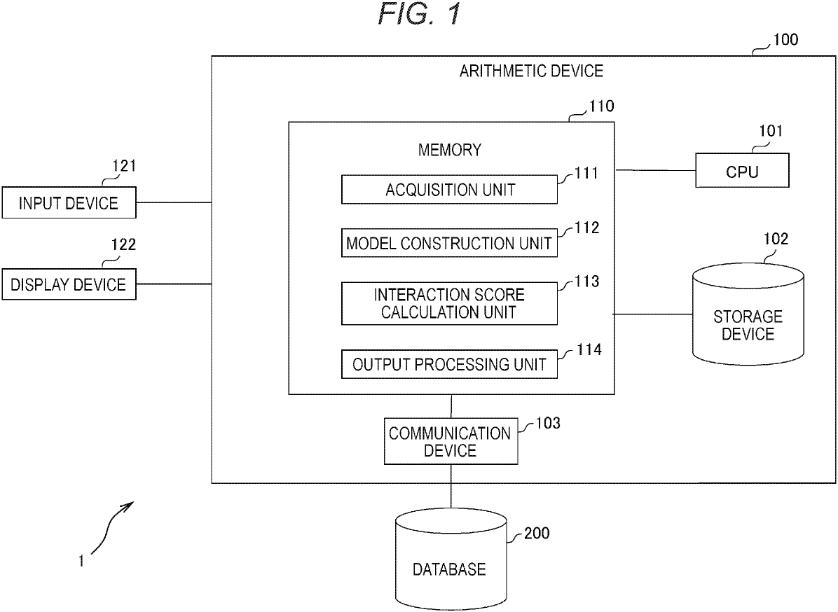

FIG. 1 is a diagram showing a configuration example of an arithmetic system 1 according to the present embodiment.

The arithmetic system 1 includes an arithmetic device 100 and a database 200.

The arithmetic device 100 includes a central processing unit (CPU) 101, a storage device 102 such as a hard disk (HD), a communication device 103, and a memory 110.

The program stored in the storage device 102 is loaded into the memory 110. Then, the CPU 101 executes the loaded program. As a result, an acquisition unit 111, a model construction unit 112, an interaction score calculation unit 113, and an output processing unit 114 are embodied. Further, an input device 121 such as a keyboard, a mouse, and the like and a display device 122 are connected to the arithmetic device 100.

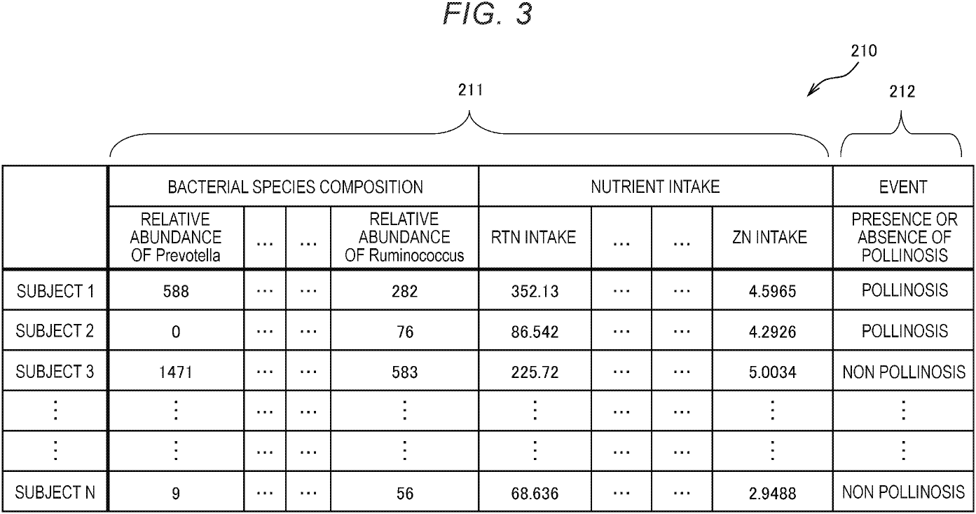

The acquisition unit 111 acquires feature amount vector data 211 (see FIG. 3) and event data 212 (see FIG. 3) required for calculating the interaction score from the database 200. The feature amount vector data 211 corresponds to the explanatory variable of the classification and prediction model, and the event data 212 corresponds to the objective variable of the classification and prediction model. The interaction score will be described later.

The model construction unit 112 constructs a classification and prediction model with a tree structure using a random forest or the like based on the acquired feature amount vector data 211 and event data 212.

The interaction score calculation unit 113 calculates the interaction score based on the classification and prediction model with a tree structure constructed by the model construction unit 112. The method of calculating the interaction score will be described later, but the interaction score is a scored degree of association of the interaction between the feature amounts with the event, based on the position of the feature amount appearing in the node that constitutes the classification and prediction model with a tree structure.

The output processing unit 114 displays the calculated interaction score on the display device 122.

The communication device 103 is connected to the database 200, receives the information of the database 200, and transmits the received information to the memory 110.

The training data 210 (see FIG. 3) is stored in the database 200. The training data 210 will be described later.

The arithmetic system 1 may be in the form of a cloud service by using the arithmetic device 100 as a cloud server.

### Flowchart

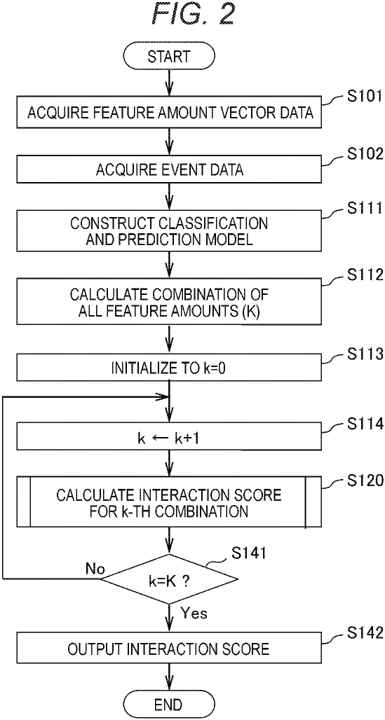

With reference to FIG. 2, an example of the processing of outputting a scored degree of association of the interaction between factors with pollinosis based on gut microbiota data and ingested nutrient data will be described.

FIG. 2 is a flowchart showing a procedure of the entire processing performed in the first embodiment.

First, the acquisition unit 111 acquires, from the database 200, feature amount vector data 211 (see FIG. 3) including information on the gut microbiota structure and ingested nutrients of the subject group stored in the database 200 (S101). The feature amount vector data 211 will be described later.

Further, the acquisition unit 111, from the database 200, acquires event data 212 (see FIG. 3) including information on the presence or absence of pollinosis in the subject group (S102). The event data 212 will be described later.

Next, the model construction unit 112 constructs a classification and prediction model with a tree structure that classifies and predicts persons with pollinosis and persons without pollenosis based on the feature amount vector using the feature amount vector data 211 and the event data 212 (S111). The classification and prediction model with a tree structure can be constructed by any algorithm, including decision trees, random forests, gradient boosting decision trees, and the like. In the present embodiment, a random forest is used.

After that, the interaction score calculation unit 113 calculates a combination of all the feature amounts (K) based on the feature amount vector (S112). The interaction score calculation unit 113 temporarily stores the number of combinations of all the feature amounts as K in the memory 110.

Subsequently, the interaction score calculation unit 113 initializes k indicating the combination number to “0” (k = 0: S113).

Then, the interaction score calculation unit 113 adds “1” to k (k ← k + 1: S114).

Next, the interaction score calculation unit 113 calculates the interaction score for the k-th feature amount combination (S120). The method of calculating the interaction score will be described later.

Subsequently, the interaction score calculation unit 113 determines whether or not k = K (S141). K is the total number of combinations of feature amounts. That is, in step S141, the interaction score calculation unit 113 determines whether or not the interaction score has been calculated for all the combinations of the feature amounts.

When the interaction score has not been calculated for all the combinations of the feature amounts (S141 - No), the interaction score calculation unit 113 returns the process to step S114.

When the interaction score is calculated for all the combinations of feature amounts (S141 → Yes), the output processing unit 114 outputs the interaction score for the predetermined combination of feature amounts to the display device 122 (S142).

### Database 200

FIG. 3 is a diagram showing an example of the training data 210 in the first embodiment.

The training data 210 is stored in the database 200 and has information on bacterial species composition, information on nutrient intake, and information on events. The information on the bacterial species composition is the structure of the gut microbiota in each subject, and specifically, the relative abundance of each intestinal bacterium is stored. The nutrient intake information stores the intake of nutrients ingested by the subject. In addition, the event information stores information on whether the subject has pollinosis (a predetermined category based on a qualitative variable having a nominal scale). A qualitative variable is a variable whose value is discrete, such as sex, name, “1st, 2nd, 3rd” and the like. In addition, the nominal scale is a scale in which only the differences in categories such as sex and name are shown, and the order between the categories is meaningless. By the way, the scale is a classification standard based on the nature of the data.

Information on the bacterial species composition of the gut microbiota can be obtained, for example, by meta 16S analysis of the gut microbiota genome. In addition, information on the bacterial species composition of the gut microbiota may be obtained from the gene composition or the like obtained from the metagenomic analysis. Further, as the information on the ingested nutrients, the food intake may be used in addition to the nutrient intake. Food intake is collected using a brief self-administered dietary history questionnaire (BDHQ) or the like. Nutrient intake can be calculated by a dedicated calculation program using BDHQ.

Of the information, regarding the bacterial species composition and nutrient intake, the numerical values for (relative abundance of Prevotella) ... (relative abundance of Ruminococcus), (intake of RTN), (intake of Zn) are listed for each subject. Such numerical values are called feature amounts, and a list of numerical values is called a feature amount vector. The information on the bacterial species composition and the ingested nutrients is the feature amount vector data 211 in FIG. 3. Further, the information about the event (whether it is pollinosis or not) is the event data 212 in FIG. 3. In this way, the event data 212 can be classified into a predetermined category by a qualitative variable having a nominal scale. That is, the feature amount vector data 211 is an explanatory variable of the classification and prediction model, and the event data 212 is the objective variable of the classification and prediction model.

### Interaction Score Calculation Processing

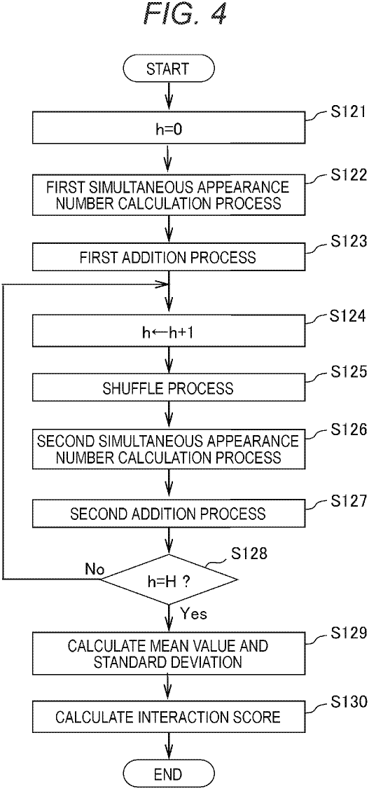

FIG. 4 is a flowchart showing the procedure of the interaction score calculation processing executed in the first embodiment. FIG. 4 shows the detailed procedure of step S120 of FIG. 2.

First, the interaction score calculation unit 113 substitutes “0” into the variable “h” indicating the current number of shuffles (S121).

Next, the interaction score calculation unit 113 performs a first simultaneous appearance number calculation process (S122). In step S122, the interaction score calculation unit 113 calculates the number of times that two feature amounts appear simultaneously in the same search branch in the decision tree in the classification and prediction model constructed in step S111 of FIG. 2. In the decision tree, the number of times that two feature amounts appear simultaneously in the same search branch is hereinafter referred to as the number of simultaneous appearances. The search branch and the number of simultaneous appearances will be described later.

Then, the interaction score calculation unit 113 performs a first addition process based on the result of the first simultaneous appearance number calculation process (S123). In step S123, the interaction score calculation unit 113 adds up the number of simultaneous appearances calculated in step S122 for the entire classification and prediction model.

Subsequently, the interaction score calculation unit 113 adds 1 to h and substitutes it for h (h ← h + 1: S124).

Then, the interaction score calculation unit 113 shuffles the classification and prediction model (S125). In step S125, the interaction score calculation unit 113 randomly shuffles the positions of the feature amounts while maintaining the topology of the decision tree. Shuffle will be described later.

Next, in step S126 in which the interaction score calculation unit 113 performs a second simultaneous appearance number calculation process (S126), the interaction score calculation unit 113 performs the same process as step S122 for the classification and prediction model subjected to the shuffle process. As a result, the interaction score calculation unit 113 calculates the number of times that two feature amounts appear simultaneously in the same search branch in the shuffle-processed classification and prediction model.

Subsequently, the interaction score calculation unit 113 performs a second addition process (S127). In step S127, the interaction score calculation unit 113 adds up the number of times that two feature amounts calculated in step S126 appear simultaneously in the same search branch in the entire classification and prediction model.

Next, the interaction score calculation unit 113 determines whether or not h = H (S128). Here, H is the number of times the interaction score calculation unit 113 shuffles.

When h = H is not satisfied (S128 → No), the interaction score calculation unit 113 returns the process to step S124.

When h = H is satisfied (S128 → Yes), the interaction score calculation unit 113 calculates the mean value and standard deviation of the number of simultaneous appearances in the classification and prediction model in which the shuffle process is performed using the result of adding the results of step S27 for each shuffle and the number of shuffles (H).

After that, the interaction score calculation unit 113 calculates the interaction score using the result of step S123 and the result of step S129 (S130). The calculation of the interaction score will be described later.

### Specific Example of Interaction Score Calculation Processing

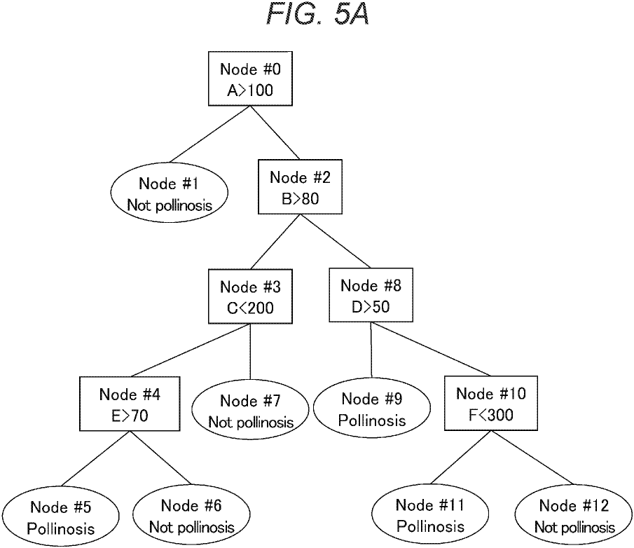

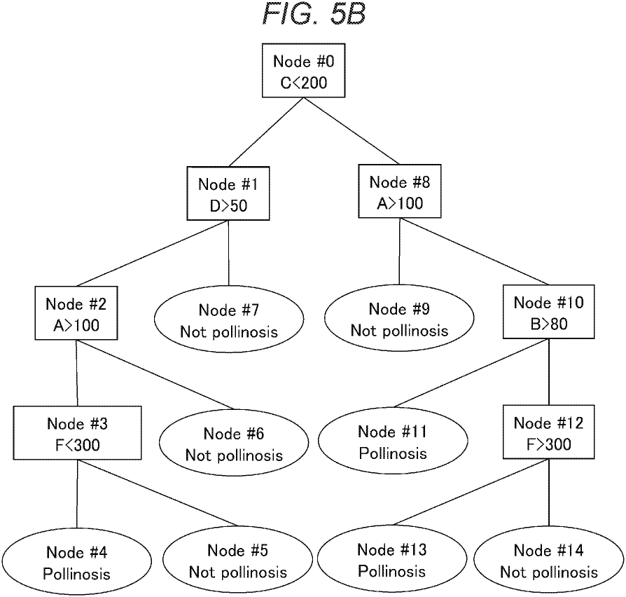

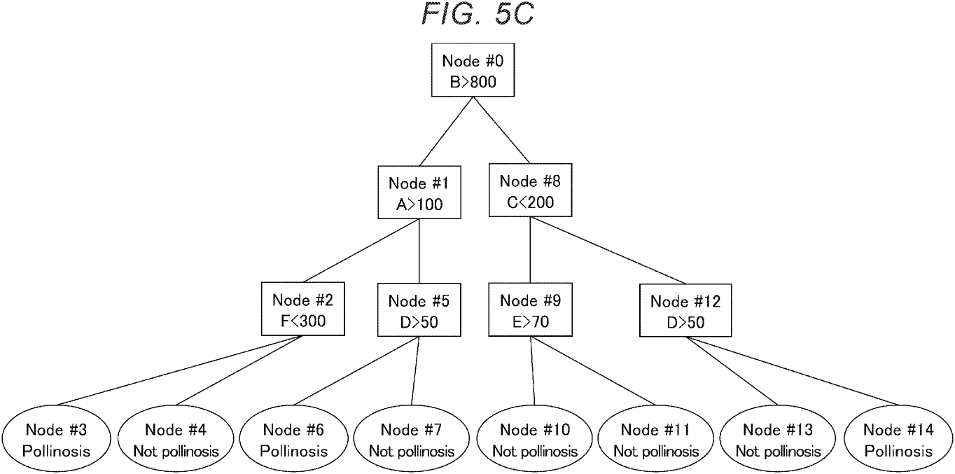

With reference to FIGS. 5A to 5C, a classification and prediction model with a random forest is shown as an example of the classification and prediction model with a tree structure for classifying and predicting the presence or absence of pollinosis, and a specific example of the interaction score calculation processing is shown.

FIGS. 5A to 5C are diagrams showing a part of the decision tree obtained as a result of applying the random forest to the training data 210.

Further, in FIGS. 5A to 5C, a total of three decision trees obtained as a result of applying the random forest to the data shown in FIG. 3 are shown.

Although three decision trees generated by the random forest are shown here, in reality, thousands to tens of thousands of decision trees constructed using randomly sampled data, and feature amounts are generated.

Further, in FIGS. 5A to 5C, “A” to “F” indicate feature amounts. That is, “A” to “F” correspond to “relative abundance of Prevotella”, “relative abundance of Ruminococcus”, “intake of RTN”, and “intake of Zn” in FIG. 3.

Further, in FIGS. 5A to 5C, the node indicated by the square is referred to as a branch node, and the terminal node indicated by the ellipse is referred to as a leaf node. A node number (#n) is assigned to each branch node and leaf node. The node number is uniquely assigned in each decision tree.

Further, the branch node located at the highest level (“Node #0” in FIGS. 5A to 5C) is referred to as a root node. Although “True” and “False” are determined in each branch node, the notation of “True” and “False” is omitted in the decision trees shown in FIGS. 5A to 5C.

Since the classification and prediction model with a tree structure such as a random forest divides the data by conditional branching, it is possible to capture the dependency between a plurality of feature amounts. Then, in the classification and prediction model with a tree structure, it has a feature that the dependency between a plurality of feature amounts is expressed in each branch of the decision tree.

Here, the branch is the route from the root node to the leaf node. For example, in the decision tree shown in FIG. 5A, the route from the root node (“Node #0”) to the leaf node “Node #12” (“Node #0”-“Node #2”-“Node #8”- “Node #10”-“Node #12”) becomes one branch.

In the route, the root node side is defined as upstream and the leaf node side is defined as downstream.

For example, in the example shown in FIG. 5A, the branch consisting of “Node #0”-“Node #2”-“Node #8”-“Node #10”-“Node #12” implicitly expresses that the interaction of the feature amounts “A”, “B”, “D”, and “F” contributes to the classification and prediction of non-pollinosis.

The intensity of the interaction between the feature amounts can be evaluated based on the number of simultaneous appearances. The number of simultaneous appearances will be described later. In the present embodiment, the intensity of the interaction between the feature amounts is shown as the interaction score. Then, in the present embodiment, the interaction score for the combination of x and y, which are any feature amounts, is defined by the following equation (1).

$$I \left(x , y\right) = \frac{N \left(x , y\right) - E \left(M \left(x , y\right)\right)}{σ \left(M \left(x , y\right)\right)}$$

In Equation (1), I(x,y) is an interaction score for a combination of x and y, which are any feature amounts. N(x,y) is the number of times (the number of simultaneous appearances) that the feature amounts x and y appear simultaneously in the same search branch in the classification and prediction model before shuffling. The search branch will be described later. Further, M(x,y) is the number of simultaneous appearances when the positions of the feature amounts are randomly shuffled while maintaining the topology of the tree. Further, E(M(x,y)) indicates the mean of M(x,y), and σ(M(x, y)) is the standard deviation of M(x,y) .

First, the calculation method of N(x,y) in Equation (1) will be described.

In the present embodiment, the search branch is defined as a route until all of the feature amounts of interest appear while following the route from the root node to the downstream.

For example, if attention is paid to the feature amounts “A” and “B” in the decision tree shown in FIG. 5A, the feature amount “A” appears in the root node “Node #0” and the feature amount “B” appears in the branch node “Node #1”. Since both the feature amounts “A” and “B” that are of interest appear in the root node “Node #0” and the branch node “Node #1”, the route downstream from “Node #1” is excluded from the search target. Therefore, in the decision tree shown in FIG. 5A, the search branch in which the feature amounts “A” and “B” appear is the route of “Node #0 - Node #1”.

Then, in this example, the number of times that the feature amounts “A” and “B” appear simultaneously in the decision tree shown in FIG. 5A is “1”.

That is, when the search branch is defined as described above, the number of times that two feature amounts appear simultaneously in the same search branch (the number of simultaneous appearances) is synonymous with calculating the number of search branches in each decision tree.

Based on the above, with reference to FIGS. 5A to 5C, N(A,F) is obtained as a specific example of N(x,y).

In the decision tree shown in FIG. 5A, the feature amount “A” appears in “Node #0”, and the feature amount “F” appears in “Node #10”. Therefore, in the decision tree shown in FIG. 5A, the search branches in which the feature amount “A” and the feature amount “F” appear are one, “Node #0” - “Node #2” - “Node #8” - “Node #10”. That is, for the decision tree shown in FIG. 5A, the number of simultaneous appearances is “1”.

In the decision tree shown in FIG. 5B, the feature amount “A” appears in “Node #2” and “Node #8”, and the feature amount “F” appears in “Node #3” and “Node #12”. Therefore, in the decision tree shown in FIG. 5B, the search branch in which the feature amount “A” and the feature amount “F” appear is two, “Node #0” - “Node #1” - “Node #2” - “Node #3”, and “Node #0” - “Node #8” - “Node #10” - “Node #12”. That is, for the decision tree shown in FIG. 5B, the number of simultaneous appearances is “2”.

Then, in the decision tree shown in FIG. 5C, the feature amount “A” appears in “Node #1”, and the feature amount “F” appears in “Node #2”. Therefore, in the decision tree shown in FIG. 5C, the search branch in which the feature amount “A” and the feature amount “F” appear is one, “Node #0” - “Node #1” - “Node #2”. That is, for the decision tree shown in FIG. 5C, the number of simultaneous appearances is “1”.

In this way, the process of calculating the number of search branches (that is, the number of simultaneous appearances) in each decision tree is the process corresponding to step S122 in FIG. 4.

N(A,F) in Equation (1) is a number in which the feature amount “A” and the feature amount “F” appear simultaneously in all the decision trees. Therefore, assuming that the decision trees shown in FIGS. 5A to 5C are all decision trees, N(A,F) is calculated as “4” by adding up the number of simultaneous appearances in each decision tree. This process corresponds to the process of step S123 in FIG. 4.

Next, M(x,y), E(M(x,y)), and σ(M(x,y)) in Equation (1) will be described.

As described above, in Equation (1), M(x,y) is the number of simultaneous appearances when the positions of the feature amounts are randomly shuffled while maintaining the topology of the tree. Further, E(M(x,y)) indicates the mean of M(x,y), and σ(M(x,y)) is the standard deviation of M(x,y) .

Here, a process of randomly shuffling the position of the feature amount while maintaining the topology of the tree (shuffle process: step S125 in FIG. 4) will be described.

A shuffle is performed based on the following rules.
(Rule #1) A shuffle is performed for each decision tree.
(Rule #2) A shuffle is performed for the feature amounts that appear in each of the branch nodes of the target decision tree.

Hereinafter, the shuffle process will be described with reference to FIGS. 5A to 5C.

In the whole decision tree shown in FIG. 5A, the relationship between the feature amount and the branch node is “A (#0), B (#2), C (#3), E (#4), D (#8), F (#10)”. Here, (#n) in parentheses indicates the number of the branch node in which the feature amount appears.

The interaction score calculation unit 113 randomly shuffles the positions of the feature amounts in “A (#0), B (#2), C (#3), E (#4), D (#8), F (#10)”. For example, it is assumed that “B (#0), D (#2), F (#3), C (#4), A (#8), E (#10)” are obtained as a result of shuffling. When such a result is obtained, the interaction score calculation unit 113 assigns the feature amount “B” to the branch node (root node) “Node #0” and assigns the feature amount “D” to the branch node “Node #2”. The interaction score calculation unit 113 also assigns other feature amounts to the branch nodes in the same manner.

Further, in the whole decision tree shown in FIG. 5B, the relationship between the feature amount and the branch node is expressed as “C (#0), D (#1), A (#2), F (#3), A (#8), B (#10), F (#12)”. Then, the interaction score calculation unit 113 randomly shuffles the positions of the feature amounts in “C (#0), D (#1), A (#2), F (#3), A (#8), B (#10), F (#12)”, and assigns the shuffle result to each branch node. The result of randomly shuffling the positions of feature amounts in “C (#0), D (#1), A (#2), F (#3), A (#8), B (#10), F (#12)” is “A (#0), C (#1), F (#2), B (#3), F (#8), A (#10), D (#12)” and “D (#0), F (#1), B (#2), F (#3), C (#8), A (#10), A (#12)”, and the like.

Similarly, in the whole decision tree shown in FIG. 5C, the relationship between the feature amount and the branch node is expressed as “B (#0), A (#1), F (#2), D (#5), C (#8), E (#9), D (#12)”. Then, the interaction score calculation unit 113 shuffles the positions of the feature amounts in “B (#0), A (#1), F (#2), D (#5), C (#8), E (#9), D (#12)”, as in the decision tree shown in FIGS. 5A and 5B, and assigns the shuffled results to each branch node. The result of shuffling the positions of feature amounts in “B (#0), A (#1), F (#2), D (#5), C (#8), E (#9), D (#12)” is “D (#0), C (#1), A (#2), E (#5), B (#8), D (#9), F (#12)” and the like.

Such shuffling creates a state in which the information on the dependency between feature amounts is lost in the decision tree.

Subsequently, the interaction score calculation unit 113 calculates the number in which the feature amount “A” and the feature amount “F” appear simultaneously in the same search branch (the number of simultaneous appearances) for each decision tree in which the result of shuffling the position of the feature amount is assigned. This process is performed in the same manner as before the shuffling process. Incidentally, this process corresponds to step S126 in FIG. 4.

Then, the interaction score calculation unit 113 adds up the number of simultaneous appearances obtained for each decision tree in all the decision trees. This result is M(A,F) of Equation (1). This process corresponds to step S127 in FIG. 4.

The interaction score calculation unit 113 performs such shuffling a plurality of times (for example, about 10 times). Then, the interaction score calculation unit 113 divides the accumulation of M(A,F) for each shuffle by the number of shuffles to calculate E(M(A,F)) of Equation (1), which is the mean value of M(A,F). Further, the interaction score calculation unit 113 calculates σ(M(A,F)) of Equation (1), which is the standard deviation of M(A,F) based on M(A,F) and E(M(A,F)). This process corresponds to step S129 in FIG. 4.

Subsequently, the interaction score calculation unit 113 substitutes the calculated M(A,F), E(M(A,F)), and σ(M(A,F)) into Equation (1) to calculate I(A,F) (interaction score). This process corresponds to step S130 in FIG. 4.

The interaction score calculation unit 113 calculates the interaction score for each combination of all the feature amounts (corresponding to steps S114 to S141 in FIG. 2) .

In Equation (1), normalization is performed according to the state in which the information on the dependency between the feature amounts is lost ((M(x,y)). In this way, by normalizing the state in which the information on the dependency between feature amounts is lost, the intensity of the interaction is well reflected.

N(A,F) indicates the number of simultaneous appearances in each decision tree generated by model construction. The number of simultaneous appearances indicates the intensity of the interaction between the feature amount “A” and the feature amount “B” in the decision tree. However, if the feature amount “A” and the feature amount “F” simply appear in large numbers in each decision tree, the value of N(A,F) becomes large. That is, if the feature amount “A” and the feature amount “F” simply appear in large numbers in each decision tree, even if there is little interaction between the feature amount “A” and the feature amount “F”, N(A,F) becomes large. That is, N(A,F) includes the number in which the feature amount “A” and the feature amount “F” appear at the same time in the search branch by chance.

Therefore, in the present embodiment, the number of simultaneous appearances (M(A,F)) in the state where the information on the dependency between the feature amounts is lost in each decision tree by the shuffle process is subtracted from N (A, F) . That is, M(A,F) indicates the number in which the feature amount “A” and the feature amount “F” happen to appear in the same search branch.

Therefore, the result of subtracting M(A,F) from N(A,F) shows the value (intensity) of the true interaction between the feature amount “A” and the feature amount “F”. However, since the value of M(A,F) changes depending on the result of shuffling, E(M(A,F)) in which the sum of M(A,M) with respect to the number of shuffles is divided by the number of shuffles is used by performing shuffling a plurality of times.

Furthermore, in Equation (1), data with different scales can be compared by dividing by a(M(x,y)). However, the division by σ(M(x,y)) may not be performed in Equation (1) .

By using the interaction score shown in Equation (1), the interaction between the feature amounts having a high interaction score in the classification and prediction of pollinosis patients and non-pollinosis patients, that is, the interaction between the feature amounts having a high degree of association with pollinosis can be extracted. As for the interaction score as shown in Equation (1), the interaction score can be similarly calculated for a combination of two or more any number of feature amounts.

### Example of Interaction Score Calculation Result

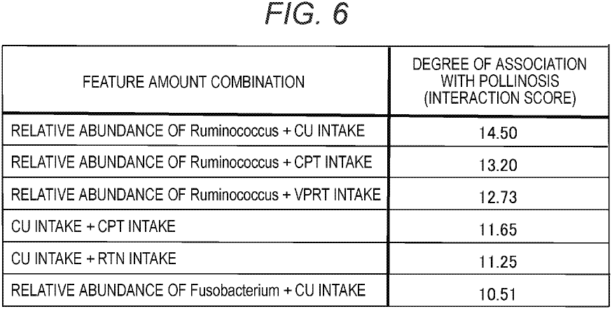

FIG. 6 is a diagram showing an example of the calculation result of the interaction score for the combination of the two feature amounts.

In the results shown in FIG. 6, the combination of feature amounts and the degree of association with pollinosis are shown in association with each other. The degree of association with pollinosis is the interaction score. That is, the higher the interaction score, the higher the degree of association with pollinosis, and the lower the interaction score, the lower the degree of association. The high degree of association with pollinosis indicates that the combination of the corresponding feature amounts is likely to be associated with the presence or absence of the onset of pollinosis.

In the example shown in FIG. 6, the combination of the intake of “Ruminococcus” as an intestinal bacterium and the intake of “Cu (copper)” as the intake of nutrients shows the highest degree of association (interaction score) with pollinosis. Incidentally, in the example shown in FIG. 6, among a large number of combinations of feature amounts, those having an interaction score of 10 or more are shown.

### Output Screen 500

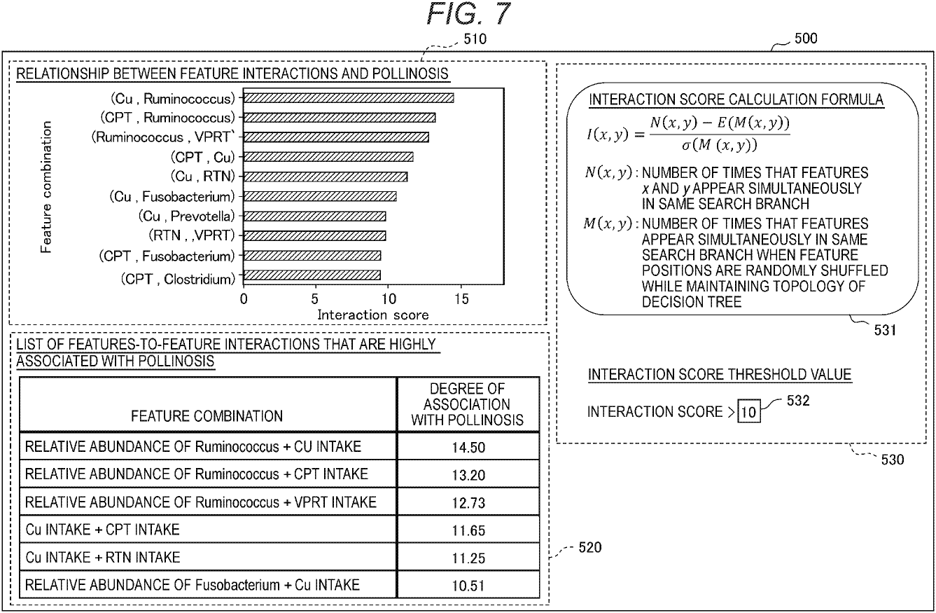

FIG. 7 is a diagram showing an example of the output screen 500 in the first embodiment. The output screen 500 shown in FIG. 7 is output in step S142 of FIG. 2.

As shown in FIG. 7, the output screen 500 has a graph display area 510, a list display area 520, and a description and setting area 530.

In the graph display area 510, the interaction score is shown as a bar graph, and the combination of feature amounts is shown in the order of the degree of association (interaction score) (ascending order). The combination of the feature amounts is shown in the graph display area 510 in the form of “(Cu, Ruminococcus)” or the like.

In the list display area 520, the combination of feature amounts and the degree of association with pollinosis (interaction score) are shown in ascending order. The display contents of the list display area 520 are the same as those in FIG. 6. That is, among a large number of combinations of feature amounts, those having an interaction score of “10” or more are shown. The combination of the feature amounts displayed in the list display area 520 is set by the threshold value setting window 532 of the description and setting area 530.

The description and setting area 530 has a calculation formula explanation area 531 and a threshold value setting window 532.

In the calculation formula explanation area 531, an explanation regarding the calculation formula of the interaction score is displayed. The calculation formula explanation area 531 can be omitted.

In the threshold value setting window 532, the threshold value of the interaction score displayed in the list display area 520 is set as described above. As described above, in the example shown in FIG. 7, since “10” is set in the threshold value setting window 532, in the list display area 520, combinations of feature amounts having an interaction score of “10” or more are shown in ascending order of the interaction score (degree of association). In the present embodiment, the threshold value set in the threshold value setting window 532 is applied to the display of the list display area 520, but it may be applied to the display of the graph display area 510. Further, the threshold value is set in advance, that is, the default setting value is set as the initial value, and the user may set the threshold value via the threshold value setting window 532.

As described above, the output screen 500 in the present embodiment can present a list of the interactions between feature amounts having a high degree of association extracted by a predetermined threshold value (threshold value set by default) or a threshold value specified by the user in the threshold value setting window 532.

According to the first embodiment, only a combination of highly associated feature amounts extracted by a method using a classification and prediction model having a tree structure (random forest in the example shown in the first embodiment) can be analyzed. This makes it possible to avoid multiple tests, which is a problem in statistical methods. That is, according to the example shown in FIG. 7, the combination of “Ruminococcus” and “Cu” having the highest degree of association (interaction score) may be analyzed. Therefore, since it is not necessary to analyze many combinations of feature amounts, multiple tests can be avoided.

In addition, since the importance used in a general classification and prediction model with a tree structure is evaluated in the presence of all other feature amounts, it is an index that also takes into account the effects of interactions between feature amounts. However, since the importance is calculated for each feature amount, the information on the interaction with respect to the combination of feature amounts is not given. On the other hand, according to the interaction score in the present embodiment, it is possible to obtain information on the interaction with respect to the combination of feature amounts. That is, the interaction score according to the present embodiment can directly evaluate what kind of interaction between feature amounts is important in classification and prediction.

In this embodiment, the nutrient intake is used as the feature amount, but the health information obtained by the health examination may also be used as the feature amount. In this case, health information may be used as a feature amount instead of the nutrient intake, or both the nutrient intake and the health information may be used as a feature amount.

As described above, in the first embodiment, a classification and prediction model with a tree structure is used based on various metadata such as information on the bacterial species composition of the gut microbiota, information on nutrient intake, health information, and the like to analyze the association between the gut microbiota and disease in consideration of the interactions between numerous feature amounts. Then, as a result, the interaction between the feature amounts associated with the disease can be extracted. That is, in the first embodiment, the degree of association of the interaction between the feature amounts with the phenotype (event) is converted into a score as an interaction score and output by using the classification and prediction model having a tree structure. This makes it possible to extract the interaction between feature amounts related to the phenotype (event). As a result, in the association analysis between the gut microbiota and the disease (presence or absence of pollinosis in the first embodiment), the interaction of the feature amount highly associated with the disease can be extracted.

### Second Embodiment

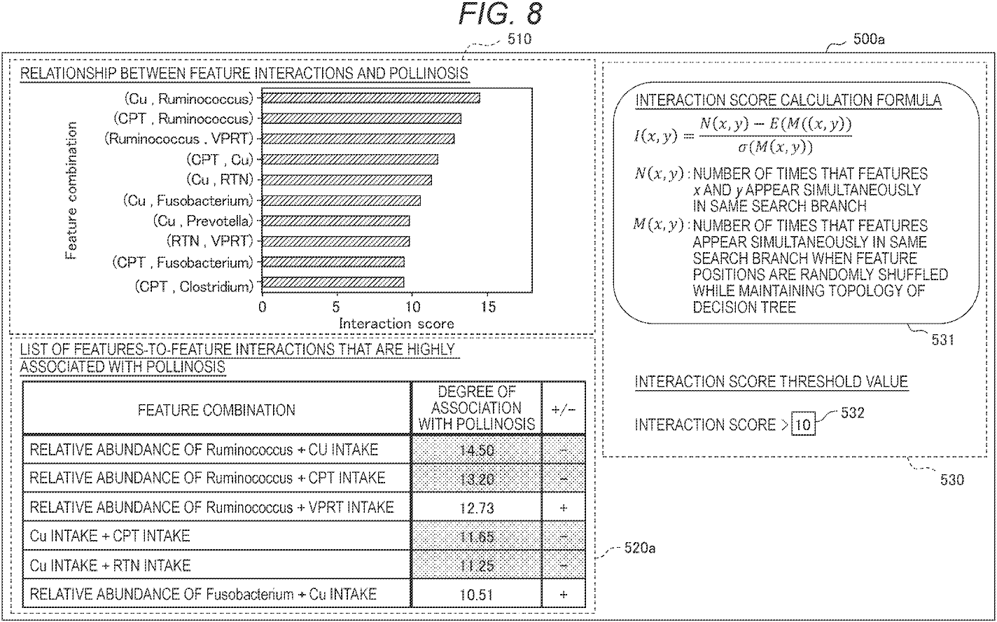

Next, a second embodiment of the present invention will be described with reference to FIG. 8.

FIG. 8 is a diagram showing an example of an output screen 500a in the second embodiment. In FIG. 8, the same components as those in FIG. 7 are designated by the same reference numerals, and the description thereof will be omitted.

By combining the machine learning method with another statistical method, it is possible to evaluate whether the interaction between the highly associated feature amounts extracted by the machine learning method is positively or negatively associated with pollinosis. In the positive association, the higher the value, the higher the probability of pollinosis, and in the negative association, the smaller the value, the higher the probability of pollinosis.

For example, by examining the sign of the coefficient corresponding to each feature amount using logistic regression in addition to the random forest, it is possible to evaluate whether the association between each feature amount and pollinosis is positive or negative. However, the method is not limited to this method, and a plurality of other statistical methods may be combined. The explanatory variable used for logistic regression is the feature amount vector data 211.

The output screen 500a shown in FIG. 8 shows an example in which the logistic regression method is applied in addition to the random forest in the list display area 520a.

In the list display area 520a of FIG. 8, a column of “+/-” is added. In the column of “+/-”, the sign of the coefficient corresponding to each feature amount is shown in the logistic regression method. In the logistic regression method, a plurality of coefficients are calculated, but if the coefficient having a negative sign is larger than the coefficient having a positive sign, “-” is stored in the “+/-” column. On the contrary, if the coefficient having a positive sign is larger than the coefficient having a negative sign, “+” is stored in the “+/-” column. If “+” is stored in the “+/-” column, it indicates that the association between each feature amount and pollinosis is positive. Further, if “-” is stored in the “+/-” column, it indicates that the association between each feature amount and pollinosis is negative.

By the way, if the coefficient having a positive sign and the coefficient having a negative sign are the same numbers, “0” is stored in the “+/-” column. In this case, it means that it is not possible to evaluate whether the association between each feature amount and pollinosis is positive or negative.

Further, in the list display area 520a, the combination of the feature amounts related to the negative is shown in shading, and the combination of the feature amounts related to the positive is shown without shading. Incidentally, the numerical value of the combination of the feature amounts displayed in the list display area 520a and the degree of association (interaction score) with pollinosis is the same as that shown in the list display area 520 of FIG. 7.

According to the second embodiment, it is possible to show the relationship between the combination of feature amounts and the probability of developing a symptom.

In the second embodiment, the random forest and the logistic regression are combined, but the analysis combined with the random forest is not limited to the logistic regression as long as it is regression analysis. For example, the random forest and multiple regression analysis may be combined.

### Third Embodiment

In the first embodiment and the second embodiment, event data 212 is used in which events such as the presence or absence of disease (presence or absence of pollinosis) can be classified into a predetermined category (predetermined category based on a qualitative variable having a nominal scale).

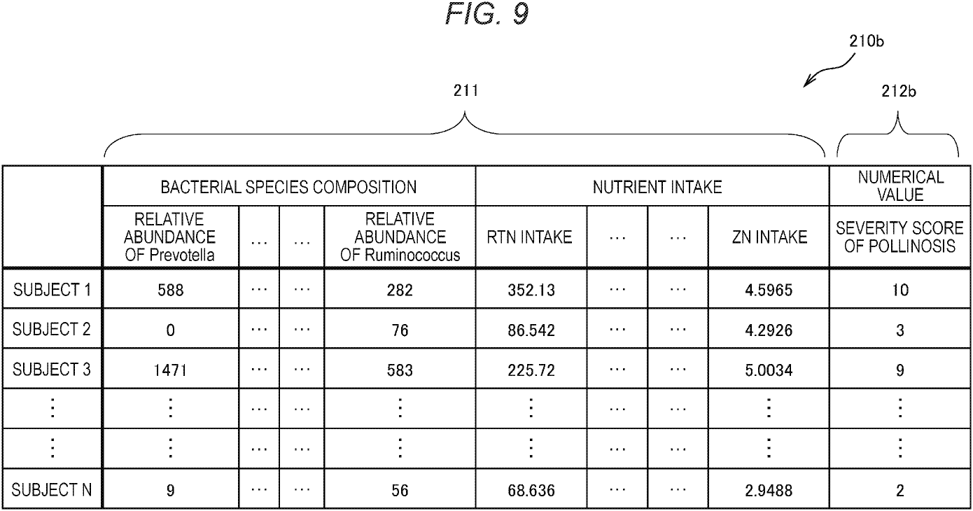

On the other hand, in the third embodiment, important interactions are extracted in the analysis for predicting some numerical values indicating the health condition of the patient. In such a case, as shown in FIG. 9, the data including the information of the numerical value of the subject group as the event data 212b is acquired together with the feature amount vector data 211 of the subject group, and a classification and prediction model with a tree structure that predicts the numerical value based on them is constructed. After that, the interaction score calculation unit 113 calculates the interaction score for each combination of the feature amounts in the same manner as the above-mentioned method at the time of classification and prediction. Finally, the output processing unit 114 outputs the interaction score.

Hereinafter, a specific example of the third embodiment will be described with reference to FIG. 9.

FIG. 9 is a diagram showing an example of training data 210b that stores feature amount vectors and numerical information of a subject group in the third embodiment.

FIG. 9 is the same as FIG. 3 except that “event”: “presence or absence of pollinosis” in FIG. 3 is “numerical value”: “severity score of pollinosis”.

That is, the training data 210b shown in FIG. 9 stores data on the severity score of pollinosis determined by the doctor for each subject based on the bacterial species composition as information on the gut microbiota structure, the intake of each nutrient as information on the ingested nutrients, and the interview as numerical information. In the example shown in FIG. 9, the severity score of pollinosis is shown on a scale of 10. However, the severity score of pollinosis is not limited to 10 levels. Thus, in the example shown in FIG. 9, the event data 212b has a qualitative variable having an ordinal scale as a numerical value. By the way, the ordinal scale is a scale in which the order between categories is meaningful, such as “1st, 2nd, 3rd”, “excellent, good, acceptable”, and the like.

That is, in the training data shown in FIG. 9, the information on the bacterial species composition and the nutrient intake becomes the feature amount vector data 211, and the numerical information becomes the event data 212b.

The model construction unit 112 (see FIG. 1) constructs a classification and prediction model for predicting the severity score of pollinosis from the feature amount vector data 211b shown in FIG. 9. The random forest or the like is used to construct the classification and prediction model. Then, the interaction score calculation unit 113 calculates the interaction score by performing the processing shown in FIG. 4, and the output processing unit 114 displays the interaction score on the display device 122.

According to the third embodiment, the same effect as that of the first embodiment can be obtained even for an event having a (discrete) numerical value such as a severity score of pollinosis.

In the example shown in FIG. 9, the severity score of pollinosis is used as the event data 212b, but it is not limited to the severity score of pollinosis as long as the regression model can be applied. In the example of the present embodiment, one ranked for the symptom of pollinosis (stuffy nose, and the like) may be used instead of the severity score of pollinosis. Alternatively, the tendency of improvement of symptoms by the drug (“excellent”, “good”, and “no change”) or the like may be used. Further, in the example shown in FIG. 9, a qualitative variable having an ordinal scale is used as the event data 212b, but a so-called quantitative data having continuous values such as blood glucose level, body weight, and BMI may be used as the event data 212b (numerical value).

Further, the second embodiment and the third embodiment may be combined.

Further, in the present embodiment, the case of the combination of two feature amounts is described in the calculation of the interaction score, but the combination of three or more feature amounts is also possible.

The present invention is not limited to the above-described embodiments and includes various modifications. For example, the above-described embodiments have been described in detail in order to explain the present invention in an easy-to-understand manner and are not necessarily limited to those having all the described configurations. Further, it is possible to replace a part of the configuration of one embodiment with the configuration of another embodiment, and it is also possible to add the configuration of another embodiment to the configuration of one embodiment. Further, it is possible to add, delete, and/or replace a part of the configuration of each embodiment with another configuration.

Further, each of the above-mentioned configurations, functions, parts 111 to 114, the storage device 102, the database 200, and the like may be achieved by hardware, for example, by designing a part or all of them by an integrated circuit or the like. Further, as shown in FIG. 1, each of the above-mentioned configurations, functions, and the like may be achieved by software by interpreting and executing a program in which a processor such as the CPU 101 implements each function. The information such as programs, tables, and files that implements each function can be stored not only in a Hard Disk (HD), but also in a recording device such as a memory 110, a Solid State Drive (SSD), or a recording medium such as an Integrated Circuit (IC) card, a Secure Digital (SD) card or a Digital Versatile Disc (DVD).

Further, in each embodiment, the control lines and information lines are shown as necessary for the explanation, and not all the control lines and information lines are shown in the product. In practice, almost all configurations may be considered to be interconnected.

What is claimed is:

1. A method for calculating an interaction between feature amounts, wherein
an arithmetic unit executes
a model construction step of acquiring data including a feature amount vector which is a set of numerical values of feature amounts and is an explanatory variable, and information of an event which is an objective variable, and constructing a classification and prediction model having a tree structure for classifying and predicting the event based on the feature amount vector;
an interaction score calculation step of calculating an interaction score in which a degree of association of an interaction between the feature amounts with the event is scored based on a position of the feature amount appearing in a node constituting the classification and prediction model, and a position of the feature amount in the classification and prediction model in which the position of the feature amount appearing in the node has been shuffled; and
an output step of outputting the calculated interaction score to an output unit.

2. The method for calculating an interaction between feature amounts according to claim 1, wherein
the classification and prediction model having a tree structure is generated by a random forest, and
in the interaction score calculation step, the following steps are executed:
a first search branch number calculation step of calculating the number of search branches, which are routes to a branch node where all of the target feature amounts appear following a route from a root node to a downstream in each of decision trees generated by the random forest,
a first addition step in which the number of search branches is added for all the decision trees,
a shuffle step of shuffling the feature amounts appearing in the decision tree for each decision tree,
a second search branch number calculation step of calculating the number of search branches for each decision tree for which the shuffle was performed,
a second addition step in which the number of search branches calculated in the second search branch number calculation step is added for all the decision trees,
a mean value calculation step of repeating from the shuffle step to the second addition step a plurality of times and calculating a mean value of the results of the second addition step based on the result of the second addition step, and
a subtraction step of subtracting the result of the mean value calculation step from the result of the first addition step.

3. The method for calculating an interaction between feature amounts according to claim 2, wherein
a division step of calculating a standard deviation with respect to the result of the second addition step based on the results of the second addition step and the mean value calculation step, and dividing the result of the subtraction step by the standard deviation is executed.

4. The method for calculating an interaction between feature amounts according to claim 1, wherein
the event can be classified into a predetermined category by a qualitative variable.

5. The method for calculating an interaction between feature amounts according to claim 1, wherein
the event has a numerical value.

6. The method for calculating an interaction between feature amounts according to claim 1, wherein
whether the interaction between the feature amounts associated with the event is positively or negatively associated with the event is evaluated using the result of applying the feature amount vector to regression analysis.

7. The method for calculating an interaction between feature amounts according to claim 1, wherein
the feature amount has a flora structure of gut microbiota and at least one of ingested nutrients and health information as a feature amount.

8. The method for calculating an interaction between feature amounts according to claim 1, wherein
the event is information on a predetermined disease.

9. A system for calculating an interaction between feature amounts comprising:
a model construction unit for acquiring data including a feature amount vector which is a set of numerical values of feature amounts and is an explanatory variable, and information of an event which is an objective variable, and constructing a classification and prediction model having a tree structure for classifying and predicting the event based on the feature amount vector;
an interaction score calculation unit for calculating an interaction score in which the degree of association of the interaction between the feature amounts with the event is scored based on a position of the feature amount appearing in a node constituting the classification and prediction model, and a position of the feature amount in the classification and prediction model in which the position of the feature amount appearing in the node has been shuffled; and
an output processing unit for outputting the calculated interaction score to an output unit.
